(19) United States
(12) Patent Application Publication
(10) Pub. No.: US 2009/0093716 A1
(43) Pub. Date: Apr. 9, 2009

(54) Method and apparatus for evaluation of labor with ultrasound

(75) Inventors: Harald Deischinger, Frankemarkt (AT); Helmut Binder-Reisinger, Seewalchen (AT); Cristina Gabardi, Milano (IT); Karl-Heinz Lumpi, St. Georgen (AT)

Correspondence Address: DEAN D. SMALL;THE SMALL PATENT LAW GROUP LLP, 225 S. MERAMEC, STE. 725T, ST. LOUIS, MO, 63105, US

(73) Assignee: GENERAL ELECTRIC COMPANY

(21) Appl. No.: 11/973,212

(22) Filed: Oct. 4, 2007

(51) Int. Cl. A61B 8/00

(52) U.S. Cl. 600/443

(57) ABSTRACT

Movement of an object within an ultrasound volume may be tracked by acquiring at least first and second volumes of ultrasound data comprising imaging data representing an anatomical structure within a patient and at least a portion of an object. The second volume is acquired at a time subsequent to the first volume. A first relationship is identified between the object and the anatomical structure on a first image that is based on the first volume. A second relationship is identified between the object and the anatomical structure on a second image that is based on the second volume. Movement of the object is determined based on at least one of the first and second relationships.

### BACKGROUND OF THE INVENTION

This invention relates generally to ultrasound and more particularly, to using ultrasound imaging to determine progress during the second stage of labor.

During the second stage of labor, the fetus or baby is pushed out through the birth canal by contracting uterine muscles, also known as contractions. Each birth is different, and some may progress quickly while others appear to stall. In many births, the baby is born in the anterior position facing the woman's back. Some babies, however, face the posterior position and may have a more difficult time moving through the woman's pelvis.

It is difficult to determine the progress of the baby during the second stage of labor. A midwife, doctor or other personnel may periodically feel to determine the current position of the baby, and to detect the progress of the labor. If satisfactory progress is not being made, interventional procedures may need to be considered. However, if slow progress is being made, it may be desirable to delay a major procedure such as a cesarean section. It is difficult to adequately determine the level of progress being made and the determination is very subjective, at least in part based on the skill of the practitioner.

Therefore, a need exists for monitoring the position of the baby during the second stage of labor.

### BRIEF DESCRIPTION OF THE INVENTION

In one embodiment, a method for tracking movement of an object within an ultrasound volume comprises acquiring at least first and second volumes of ultrasound data comprising imaging data representing an anatomical structure within a patient and at least a portion of an object. The second volume is acquired at a time subsequent to the first volume. A first relationship is identified between the object and the anatomical structure on a first image that is based on the first volume. A second relationship is identified between the object and the anatomical structure on a second image that is based on the second volume. Movement of the object is determined based on at least one of the first and second relationships.

In another embodiment, an ultrasound system comprises a transducer for acquiring volumes of ultrasound data. The volumes each comprise imaging data representing a pubis within a patient and at least a portion of a fetal head. The volumes are acquired with an elapse in time there-between. The system also comprises a display and a user interface. The display displays at least one image based on the volumes. The user interface accepts input from an operator. The input is based on at least one of the pubis and the fetal head within the at least one image, and the display indicates a relationship of the fetal head to the patient based on the input.

In yet another embodiment, a method for determining progress of a fetus during labor comprises accessing a first volume of ultrasound data comprising imaging data representing a pubis within a patient and at least a portion of a fetal head. A first relationship is identified between the fetal head and the pubis on a first image that is based on the first volume. A second volume of ultrasound data comprising imaging data representing a pubis and at least a portion of the fetal head is accessed. The second volume is acquired at a time subsequent to the first volume, and the first and second volumes are aligned with respect to the pubis. A second relationship between the fetal head and the pubis is identified on a second image that is based on the second volume, and progress of the fetal head is determined based on at least one of the first and second relationships.

### BRIEF DESCRIPTION OF THE DRAWINGS

FIG. 1 illustrates an ultrasound system formed in accordance with an embodiment of the present invention.

FIG. 2 illustrates an example of progression of labor using ultrasound imaging in accordance with an embodiment of the present invention.

FIG. 3 illustrates a method for using ultrasound to track the movement of an object within a volume, such as to evaluate progress during the second stage of labor in accordance with an embodiment of the present invention.

FIG. 4 illustrates three orthogonal images based on a volume of ultrasound data in accordance with an embodiment of the present invention.

FIG. 5 illustrates an example of determining the progress of labor based on contours of the fetal head accordance with an embodiment of the present invention.

FIG. 6 illustrates an example of determining the progress of labor based on an angular relationship between a patient's anatomy and the fetal head in accordance with an embodiment of the present invention.

FIG. 7 illustrates an example of determining the progress of labor based on rotation of a midline of the fetal head in accordance with an embodiment of the present invention.

FIG. 8 illustrates images using both contours and rotation to monitor the position of the fetal head within the patient in accordance with an embodiment of the present invention.

FIG. 9 illustrates a 3D-capable miniaturized ultrasound system formed in accordance with an embodiment of the present invention.

FIG. 10 illustrates a hand carried or pocket-sized ultrasound imaging system formed in accordance with an embodiment of the present invention.

FIG. 11 illustrates a console ultrasound imaging system formed in accordance with an embodiment of the present invention.

### DETAILED DESCRIPTION OF THE INVENTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

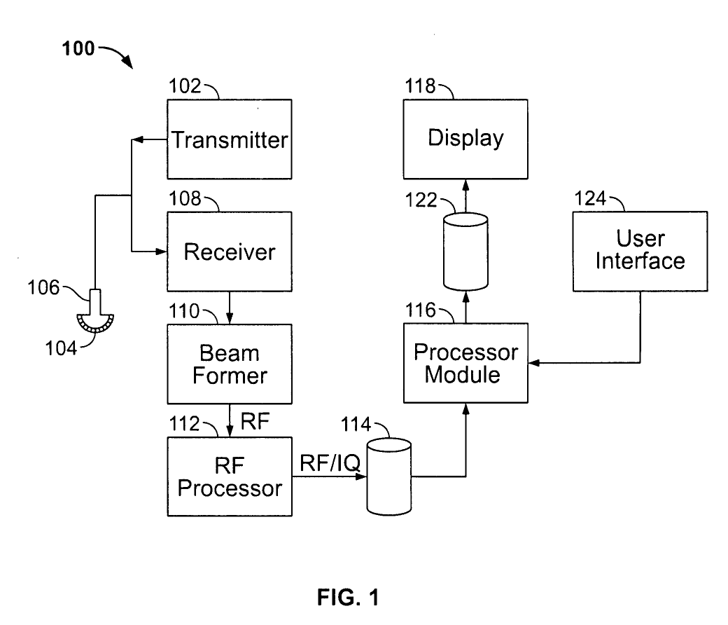

FIG. 1 illustrates an ultrasound system 100 including a transmitter 102 that drives an array of elements 104 (e.g., piezoelectric elements) within a transducer 106 to emit pulsed ultrasonic signals into a body. The elements 104 may be arranged, for example, in two dimensions. A variety of geometries may be used. The ultrasonic signals are back-scattered from structures in the body, like fatty tissue, muscular tissue and bone, to produce echoes that return to the elements 104. The echoes are received by a receiver 108. The received echoes are passed through a beamformer 110, which performs beamforming and outputs an RF signal. The RF signal then passes through an RF processor 112. Alternatively, the RF processor 112 may include a complex demodulator (not shown) that demodulates the RF signal to form IQ data pairs representative of the echo signals. The RF or IQ signal data may then be routed directly to a memory 114 for storage.

The ultrasound system 100 also includes a processor module 116 to process the acquired ultrasound information (e.g., RF signal data or IQ data pairs) and prepare frames of ultrasound information for display on display 118. The processor module 116 is adapted to perform one or more processing operations according to a plurality of selectable ultrasound modalities on the acquired ultrasound information. Acquired ultrasound information may be processed and displayed in real-time during a scanning session as the echo signals are received. Additionally or alternatively, the ultrasound information may be stored temporarily in memory 114 during a scanning session and then processed and displayed in an off-line operation.

The processor module 116 is connected to a user interface 124 that may control operation of the processor module 116 as explained below in more detail. The display 118 includes one or more monitors that present patient information, including diagnostic ultrasound images to the user for diagnosis and analysis. One or both of memory 114 and memory 122 may store three-dimensional (3D) data sets or volumes of the ultrasound data, and the volumes are accessed to present 2D and 3D images. Multiple consecutive volumes may also be acquired and stored over time, such as to provide real-time 3D or 4D display. The images may be modified and the display settings of the display 118 also manually adjusted using the user interface 124.

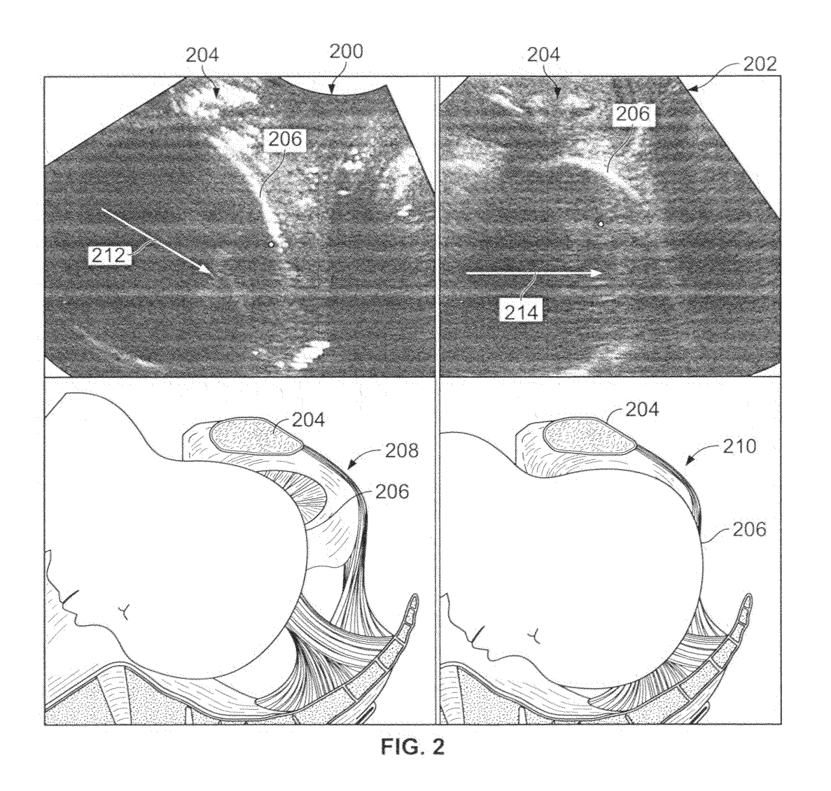

Ultrasound systems 100 are often used during a woman's pregnancy to document the progression of the pregnancy as well as to evaluate the fetus. As discussed previously, during labor medical personnel typically rely on physical examination to determine progress of the fetus, or lack thereof, through the birth canal. FIG. 2 illustrates an example of progression of labor using ultrasound imaging during the second stage of labor. First and second images 200 and 202 may be longitudinal images based on two different volumes that were acquired at different times. The first and second images 200 and 202 may be displayed on the display 118. For example, a first volume may be acquired, then a second volume may be acquired after an elapsed time, such as 10 or 15 minutes. In one embodiment, a minimum elapsed time may be set, such as 2 or 5 minutes. In one embodiment, annotations, such as arrows 212 and 214, may be used by the operator to generally indicate the direction of the movement of the fetal head 206.

Symphysis pubis (pubis) 204 within the patient and fetal head 206 are imaged within both of the first and second images 200 and 202. The pubis 204 is a bony structure and thus relatively easy to identify on the ultrasound image, even if the image is a relatively low resolution or lower quality than may be used in other scanning applications. The position of the fetal head 206 may be determined by identifying parietal bones which form the top and sides of the fetal head 206.

First and second drawings 208 and 210 are associated with the first and second images 200 and 202, respectively. The first and second drawings 208 and 210 also show the pubis 204 and fetal head 206. In the first and second drawings 208 and 210, the relationship of the pubis 204 and the fetal head 206 are easy to determine, and it can be determined that progress in labor is made from the first drawing 208 to the second drawing 210. Likewise, referring to the first and second images 200 and 202, the relationship of the pubis 204 and fetal head 206 and/or the position of the fetal head 206 from one ultrasound image to the next may be identified by an operator of the ultrasound system 100 to evaluate the progress of labor.

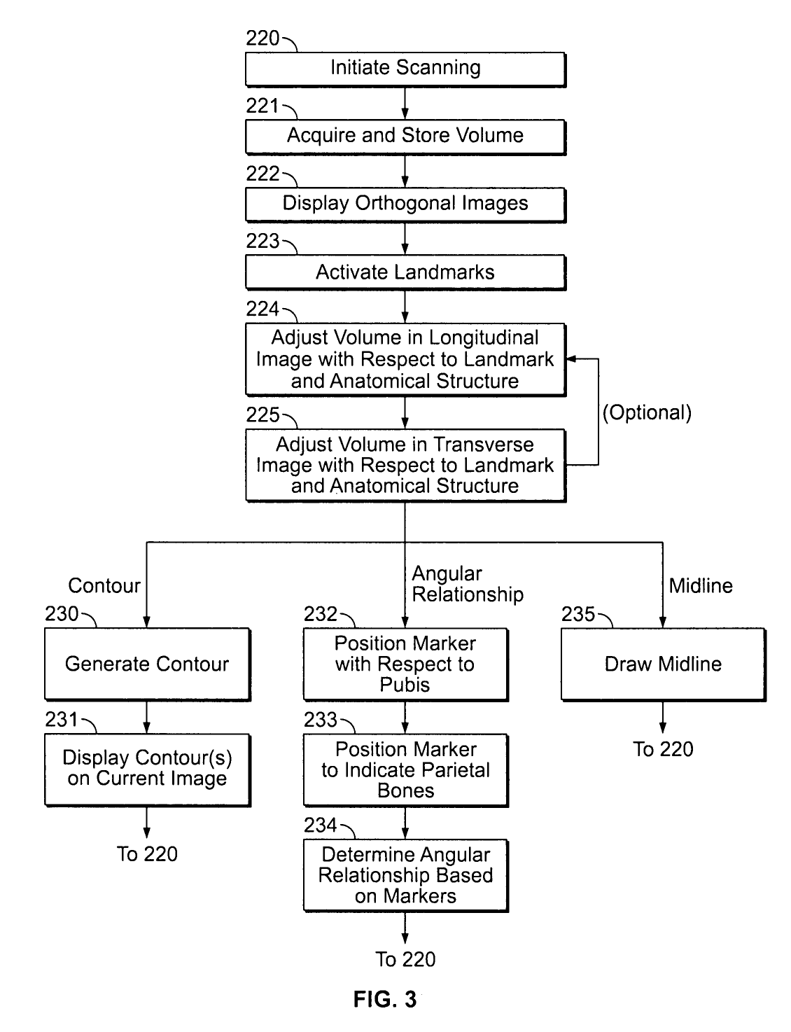

FIG. 3 illustrates a method for using the ultrasound system 100 of FIG. 1 to track the movement of an object within an ultrasound volume, such as to evaluate progress during the second stage of labor. In one embodiment, the object may be a fetal head that is tracked during labor. In another embodiment, the object may be a foreign body or other obstruction that may be tracked, such as a foreign body within a patient's digestive tract. At 220, the operator initiates scanning with the transducer 106 which is placed in infrapubic position. With the user interface 124 the operator may adjust scanning parameters such as depth and scale, which will remain constant over the acquisition of multiple volumes. It should be understood that different positioning of the transducer 106 may be used to image other structures and objects. At 221, a volume of ultrasound data is acquired and may be stored in the memory 122. Once the volume is stored, the operator may move away from the patient. Therefore, the analysis of the volume may be accomplished remote from the patient.

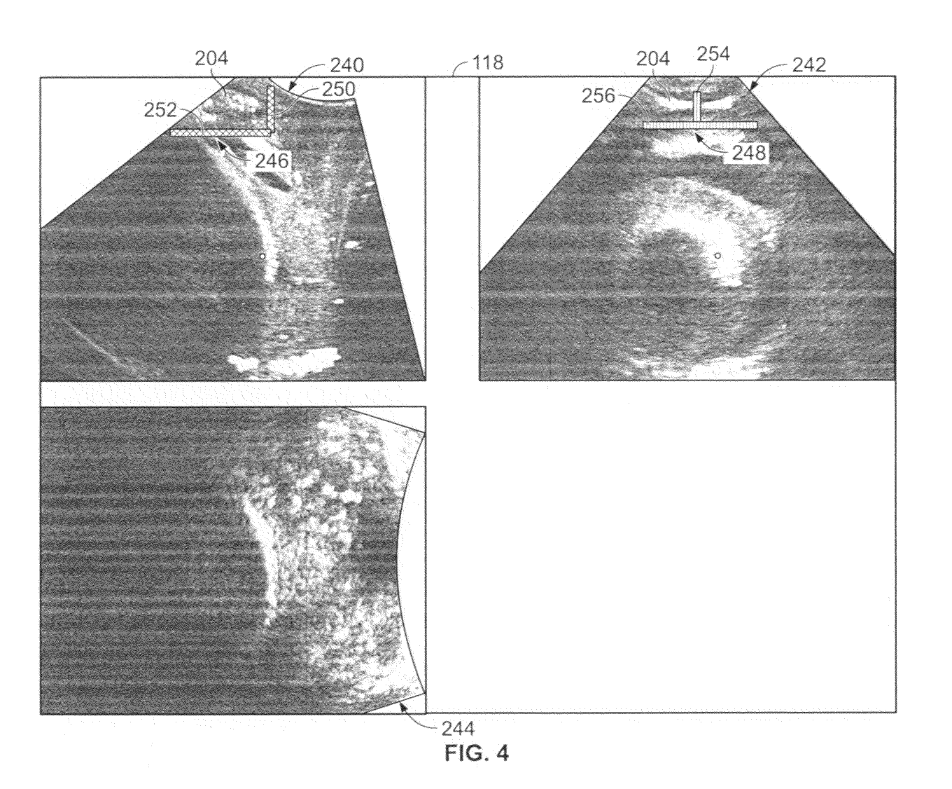

At 222, the operator displays one or more images on the display 118 that are based on the volume of 221. FIG. 4 illustrates three orthogonal images based on a volume of ultrasound data. In this example, first, second and third images 240, 242 and 244 may be displayed simultaneously, corresponding to longitudinal, transverse and coronal images, respectively. In one embodiment, the operator may toggle through the first, second and third images 240, 242 and 244 to display one image or display two selected orthogonal images simultaneously. For example, if the display 118 is large, the operator may choose to display all of the images 240-244 simultaneously, while if the display 118 is small, the operator may chose to display one image at a time.

At 223, the operator activates landmarks that are overlaid on the first and second images 240 and 242. Alternatively, the processor module 116 may automatically generate and display the landmarks. Referring again to FIG. 4, first and second landmarks 246 and 248 are overlaid on the first and second images 240 and 242, respectively. The first and second landmarks 246 and 248 are geometric landmarks; however, other shapes, colors, and the like may be used. In this example, the first landmark 246 is an “L” shape with a first portion 250 extending vertically with respect to the display 118 and located at a horizontal center of the first image 240. A second portion 252 extends perpendicular to the first portion 250 at a predetermined distance from the top of the first image 240. The second landmark 248 is a “T” shape with a first portion 254 extending vertically with respect to the display 118 and located at the horizontal center of the second image 242. A second portion 256 extends perpendicular to the first portion 254 at a predetermined distance from the top of the second image 242. In one embodiment, the first and second landmarks 246 and 248 may be repositioned and/or altered in shape or orientation by the operator, however, the first and second landmarks 246 and 248 remain constant from one acquired volume to the next, providing the operator visual cues to align volumes such that the same tissue is included from volume to volume.

At 224, the operator identifies the desired anatomical structure, in this example the pubis 204, of the patient in the longitudinal image, which is the first image 240 of FIG. 4. The operator adjusts the volume as needed, such as by rotation and/or translation, to position the pubis 204 within the first image 240 with respect to the first landmark 246. In one embodiment, the operator adjusts the volume so that the pubis 204 is to the left side of the first portion 250 of the first landmark 246 and above the second portion 252. It should be understood that if other areas of the body are being imaged, other anatomical structures may be aligned with respect to the landmark 246.

At 225, the operator identifies the pubis 204 in the transverse image, which is the second image 242 of FIG. 4. The operator adjusts the volume so that the pubis 204 is positioned within the second image 242 with respect to the second landmark 248. In one embodiment, the operator adjusts the volume so that the first portion 254 of the second landmark 248 intersects the pubis 204 along the center (not shown) of the pubis 204. Also, the volume is adjusted to position the pubis 204 above the second portion 256. It should be understood that other orientations of the patient's anatomy (e.g. the pubis or other anatomical structure) may be made to the landmarks as long as the orientation is constant from volume to volume.

In one embodiment, adjusting the volume with respect to the first and second landmarks 246 and 248 (at 224 and 225) may be repeated until the operator is satisfied with the position of the volume. This process aligns the volume in space, providing reproducible landmarks so that subsequent volumes may be similarly aligned. In this manner, even though subsequent volumes may not be acquired in exactly the same position due to movement of the patient and repositioning of the transducer 106, multiple volumes over time may be oriented in the same way and thus may be compared to each other. Depending upon the size and/or capabilities of the display 118, the operator may view both of the first and second images 240 and 242 at the same time, or may toggle back and forth between the two images until satisfied with the position of the volume. In this example, the third image 244 is not used for positioning.

Several methods may be used to determine the progress of labor based on the stored volume such as changes in the position of a contour of the fetal head 206 over time and/or an angular relationship between the patient's anatomy and the fetal head 206 over time. Also, rotation of the fetal head 206 over time may be determined. Although the discussion below is with respect to the progress of labor, the method applies equally to other anatomical structures and objects that may be tracked and compared over time within the volumes.

Alternatively, positioning the anatomical structure with respect to the landmarks may at least partially be accomplished during scanning, prior to acquiring and storing the volume at 221. For example, the operator may verify while scanning that both the anatomical structure and the object of interest, in this case the fetal head 206, (FIG. 2) are within an ultrasound image viewed on the display 118. In one embodiment, the operator may view one or more images with the landmarks overlaid thereon in real-time, adjusting the position of the transducer 106 to position the anatomical structure with respect to the landmarks. Additional adjustment, such as rotation and/or translation of the volume, may be accomplished after the volume is stored as discussed previously.

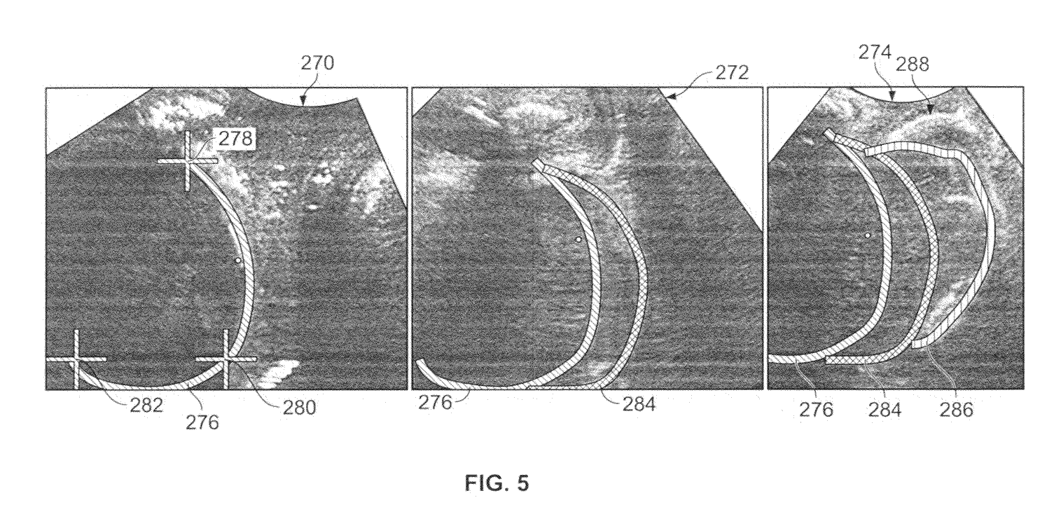

FIG. 5 illustrates an example of determining the progress of labor based on volumes of data that are acquired at different points in time. First, second and third images 270, 272 and 274 are illustrated and correspond to longitudinal images of first, second and third volumes, respectively. The volumes are not shown. For example, the second volume may be acquired 15 minutes after the first volume, and the third volume may be acquired 10 minutes after the second volume.

Returning to FIG. 3, the position of the contour of the fetal head 206 will be discussed first. At 230 the operator may use the user interface 124 to draw a first contour 276 on the first image 270. The first contour 276 may be generated by selecting one or more points along the parietal bones of the fetal head 206, such as first, second and third points 278, 280 and 282. The processor module 116 may then apply one or more of edge detection and predetermined forms to generate the first contour 276. Optionally, the operator may modify an automatically or semi-automatically generated contour by dragging the first contour 276 to better match the actual contour of the fetal head 206. Alternatively, the operator may manually draw the first contour 276. At 231, the processor module 116 displays the first contour 276 on the first image 270, such as with an overlay. The first contour 276 may be stored in a separate file in the memory 122.

After the first contour 276 is drawn, the operator may wait a period of time before acquiring a second volume. The period of time may be based on personal experience, the position of the fetal head 206 on the first image 270, the health of the patient, length of time elapsed in labor, and the like. After the period of time, the operator repeats 220-225 to acquire and align the second volume. The same imaging parameters are used to acquire both of the first and second volumes. At 224 and 225, the same first and second landmarks 246 and 248 are used. Therefore, the orientation of the patient anatomy with respect to the landmarks is the same in the first volume and the subsequent volumes.

At 230 the operator generates a second contour 284 on the second image 272. At 231, the processor module 116 displays both the first and second contours 276 and 284 on the second image 272. The first and second images 270 and 272 are based on the first and second volumes, respectively, that have the same orientation and have substantially the same patient anatomy. Therefore, the operator may compare the first and second contours 276 and 284 to determine how and if the fetal head 206 is moving with respect to the patient anatomy, thus determining whether progress has been made in labor. In another embodiment, the processor module 116 may automatically determine a difference between the first and second contours 276 and 284, such as an estimated distance between the two in centimeters, and display the difference on the display 118.

The process may be repeated any number of times, acquiring and adjusting a volume of data, drawing the contour, and comparing the current contour to one or more previous contours. For example, the third image 274 is based on a third volume. The operator generates a third contour 286, which has a less uniform curved shape than the first and second contours 276 and 284. In this example, a caput 288, or soft tissue swelling, is displayed in the third image 274, which is information that the caregiver may use when determining further course of action for the patient. The contours 276, 284 and 286 may be toggled on/off using the user interface 124, one or more of the contours 276, 284 and 286 may be removed, and all of the contours 276, 284 and 286 may be removed, allowing the operator to view the ultrasound image unobstructed. Also, the contours may be displayed using different representations, such as different colors, thickness of display lines, format of display lines and the like.

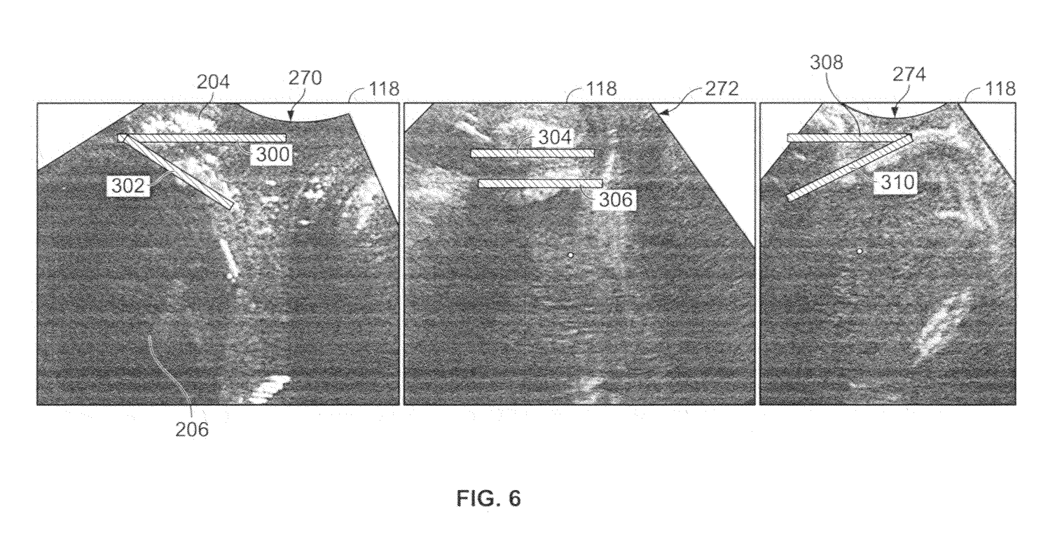

Returning to 225 of FIG. 3, in another embodiment, progression of labor may be determined based on an angular relationship between the patient's anatomy and the fetal head 206 or other object within the patient. FIG. 6 illustrates the first, second and third images 270, 272 and 274 of FIG. 5. Therefore, the same images may be used to determine the progression of labor based on one or both of an angular relationship and a contour.

At 232, the operator may position a first marker 300 with respect to the pubis 204, such as along a lower edge of the pubis 204. The first marker 300 may be aligned horizontally with respect to the display 118. At 233, the operator positions a second marker 302 with respect to the parietal bones of the fetal head 206. For example, the operator may select a point (not shown) on the first image 270 that is approximately the start of the parietal bone. The processor module 116 may then generate a line (the second marker 302) as a tangent to the selected point. Again, the operator may adjust the first and second markers 300 and 302 manually with the user interface 124. In one embodiment, the first and second markers 300 and 302 may be moved by the operator, such as by a mouse, to a desired position and orientation with respect to anatomy.

At 234, the processor module 116 determines an angular relationship based on the first and second markers 300 and 302. An angle expressed in degrees, for example, may be determined between the first and second markers 300 and 302 and indicated separately (not shown) on a side of the display 118. Therefore, the method provides the angular relationship based on the first marker 300 (which is based on the patient's anatomy) and the second marker 302 (which is based on the fetal head 206).

The angular relationship may be determined for the second and third images 272 and 274 as well by positioning first and second markers 304 and 306 on the second image 272 and first and second markers 308 and 310 on the third image 274. The change in angular relationship may be expressed along a side of the display 118 in degrees, for example, and/or an amount of change with respect to one or more previous measurements. In this example, the markers of the first, second and third images 270, 272 and 274 may be visually confusing if displayed on a single image. Therefore, two or more of the first, second and third images 270, 272 and 274 may be displayed simultaneously on the display 118. In another embodiment, a single image may be displayed while the operator toggles markers from different images on and off. For example, the operator may cycle through the markers in order based on time, displaying the progression of the labor.

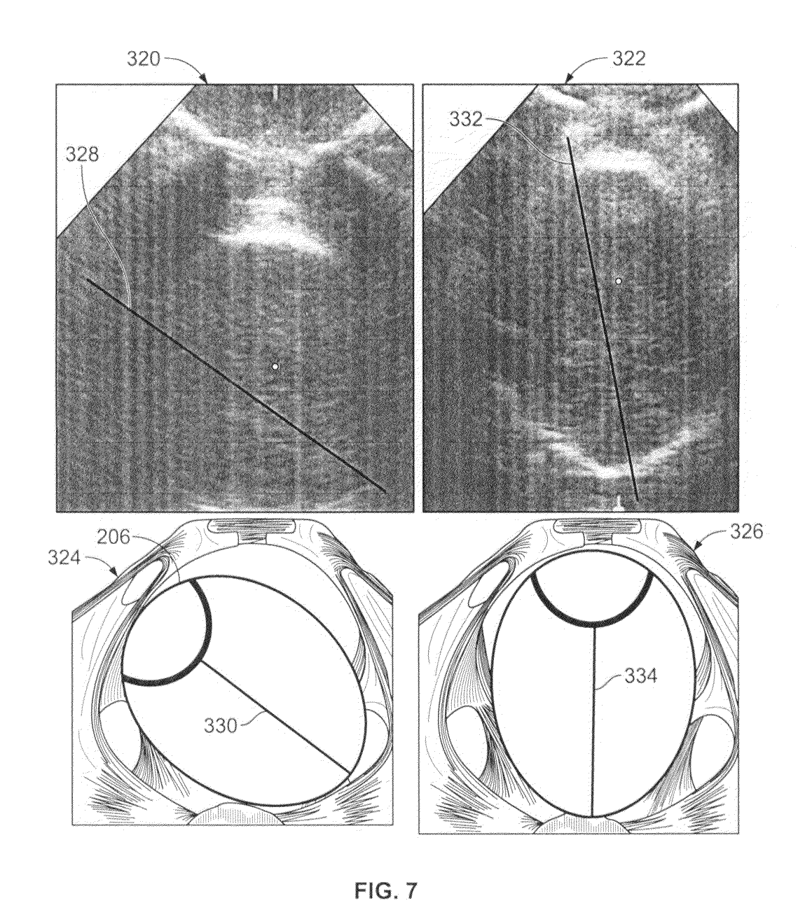

Labor may also be evaluated by detecting rotation of the fetal head 206 within the images of the volumes. FIG. 7 illustrates transverse images and corresponding schematic views of the patient anatomy and fetal head 206. First and second images 320 and 322 may be transverse images based on the first and second volumes that were acquired and adjusted as discussed in FIG. 3. First and second drawings 324 and 326 correspond to the first and second images 320 and 322, respectively.

Returning to FIG. 3, at 235 the operator may display the first image 320 (based on the first volume) and draw a first midline 328 that corresponds to a position of a first midline 330 of the fetal head 206 in the first drawing 324. Similarly, in the second image 322 based on the second volume, the operator uses the user interface 124 to draw a second midline 332 that corresponds to a position of a second midline 334 in the second drawing 326. The processor module 116 may calculate rotation angles associated with the first and second midlines 328 and 332, such as with respect to a horizontal line (not shown) that may be similar to the first marker 300 of FIG. 6. Therefore, the rotation angle may be calculated with respect to the anatomy of the patient, such as with respect to the plane of the pubis 204.

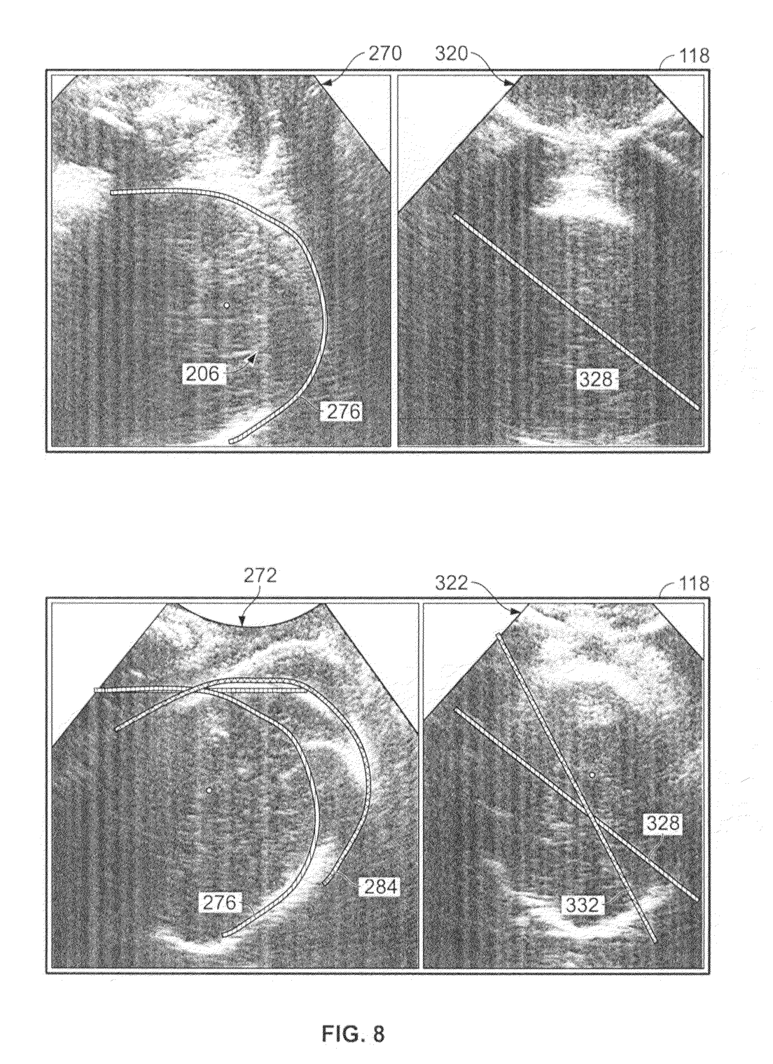

FIG. 8 illustrates an example of displaying images using both contours and rotation to monitor the position of the fetal head 206 within the patient. For example, after the first volume is acquired and adjusted at 225 of FIG. 3, the first image 270 and first contour 276 of FIG. 5 may be shown on the display 118 simultaneously with the first image 320 and the first midline 328 of FIG. 7. The two different images and markings provide information to the operator to track the progress of the labor, and the operator can verify that the rotation is moving in the desired direction.

After the second volume is acquired and stored, the second image 272 with the first and second contours 276 and 284 is shown with the second image 322 having the first and second midlines 328 and 332 overlaid thereon. The first contour 276 and first midline 328 may be displayed in a first color, line thickness, brightness and the like, while the second contour 284 and second midline 332 may be shown using a different representation. This allows the operator to easily see the progress of labor between the acquisitions of the two volumes. In another embodiment, the parietal angle measurement may also be accomplished by the operator and shown on the longitudinal images.

The first, second and subsequent volumes may be acquired using any ultrasound system, such as a miniaturized system, small-sized system, or a portable cart-based system. Therefore, the system used may be determined by the availability of the system, as well as available room for the system proximate to the patient. Also, the contours, angular measurements and midline measurements may be accomplished in the room with the patient, or may be accomplished away from the patient. The volume data may also be transferred to a different system or workstation for the measurements to be accomplished and/or compared.

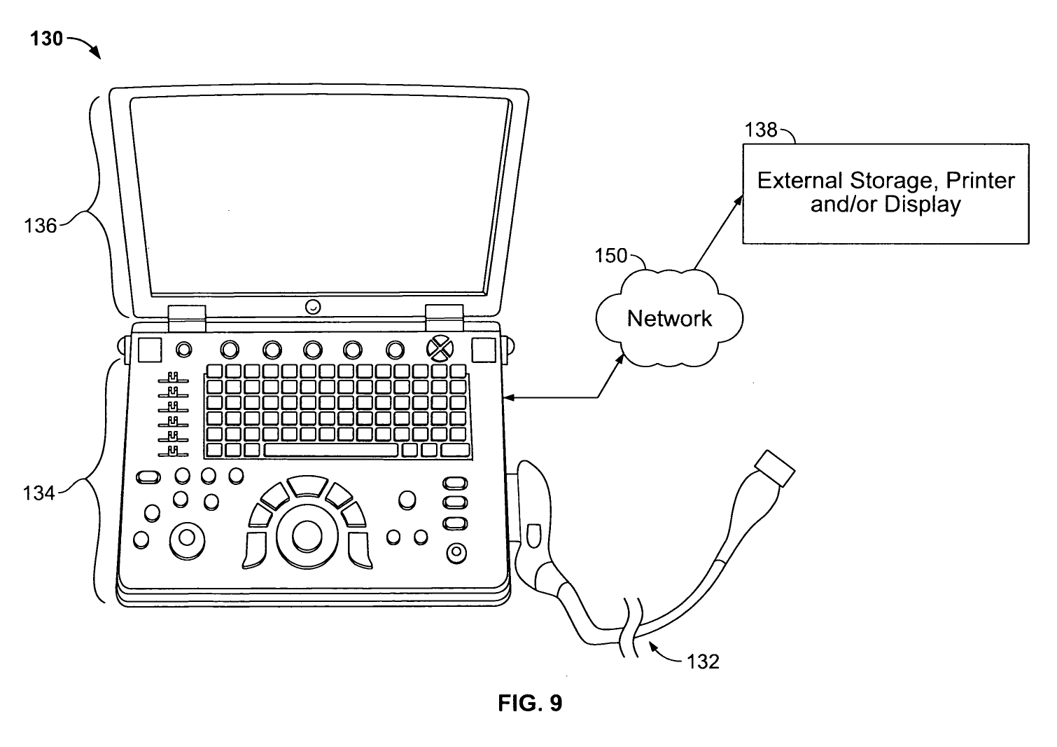

FIG. 9 illustrates a 3D-capable miniaturized ultrasound system 130 having a transducer 132 configured to acquire 3D ultrasonic data. For example, the transducer 132 may have a 2D array of transducer elements 104 as discussed previously with respect to the transducer 106 of FIG. 1. A user interface 134 (that may also include an integrated display 136) is provided to receive commands from an operator. As used herein, “miniaturized” means that the ultrasound system 130 is a handheld or hand-carried device or is configured to be carried in a person's hand, pocket, briefcase-sized case, or backpack. For example, the ultrasound system 130 may be a hand-carried device having a size of a typical laptop computer, for instance, having dimensions of approximately 2.5 inches in depth, approximately 14 inches in width, and approximately 12 inches in height. The ultrasound system 130 may weigh about ten pounds, and thus is easily portable by the operator. The integrated display 136 (e.g., an internal display) is also provided and is configured to display a medical image.

The ultrasonic data may be sent to an external device 138 via a wired or wireless network 150 (or direct connection, for example, via a serial or parallel cable or USB port). In some embodiments, external device 138 may be a computer or a workstation having a display. Alternatively, external device 138 may be a separate external display or a printer capable of receiving image data from the hand carried ultrasound system 130 and of displaying or printing images that may have greater resolution than the integrated display 136.

As another example, the ultrasound system 130 may be a 3D capable pocket-sized ultrasound system. By way of example, the pocket-sized ultrasound system may be approximately 2 inches wide, approximately 4 inches in length, and approximately 0.5 inches in depth and weigh less than 3 ounces. The pocket-sized ultrasound system may include a display, a user interface (i.e., keyboard) and an input/output (I/O) port for connection to the transducer (all not shown). It should be noted that the various embodiments may be implemented in connection with a miniaturized ultrasound system having different dimensions, weights, and power consumption.

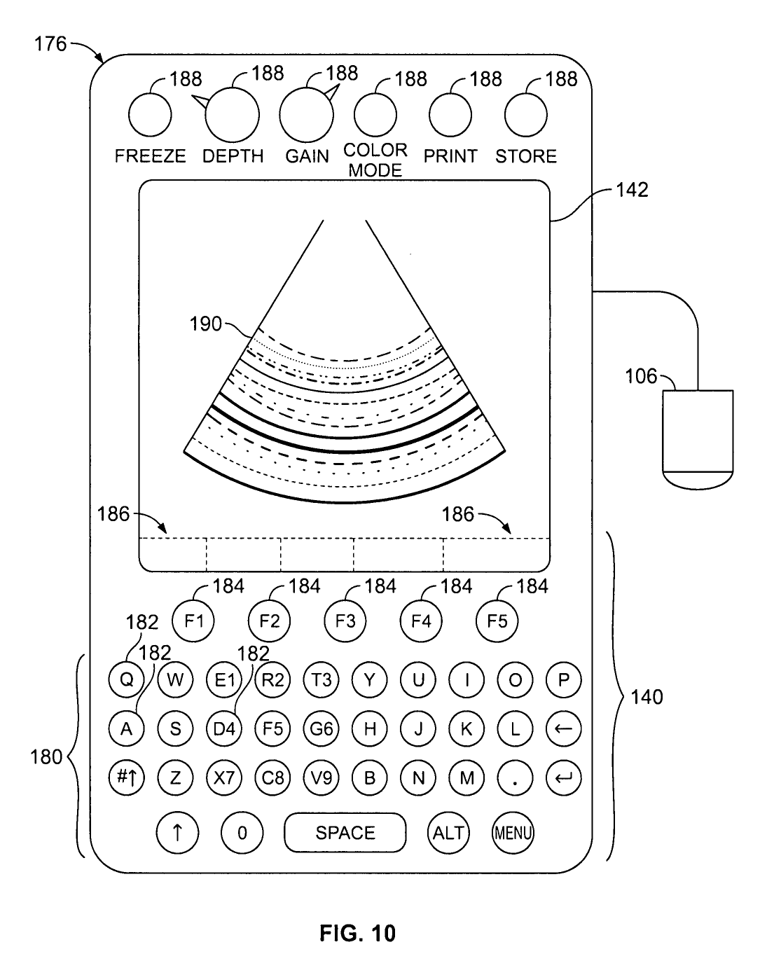

FIG. 10 illustrates a hand carried or pocket-sized ultrasound imaging system 176 wherein the display 142 and user interface 140 form a single unit. By way of example, the pocket-sized ultrasound imaging system 176 may be a pocket-sized or hand-sized ultrasound system approximately 2 inches wide, approximately 4 inches in length, and approximately 0.5 inches in depth and weighs less than 3 ounces. The display 142 may be, for example, a 320×320 pixel color LCD display (on which a medical image 190 may be displayed). A typewriter-like keyboard 180 of buttons 182 may optionally be included in the user interface 140.

Multi-function controls 184 may each be assigned functions in accordance with the mode of system operation. Therefore, each of the multi-function controls 184 may be configured to provide a plurality of different actions. Label display areas 186 associated with the multi-function controls 184 may be included as necessary on the display 142. The system 176 may also have additional keys and/or controls 188 for special purpose functions, which may include, but are not limited to “freeze,” “depth control,” “gain control,” “color-mode,” “print,” and “store.”

The small sizes of the systems in FIGS. 9 and 10 may be advantageous as they have a small footprint, are hand-carried and can be easily moved from one location to the next. In addition, small-sized systems may be used to evaluate labor in settings other than hospitals, such as in a patient's home or a birthing center, or may be used by personnel during transportation of the patient to the hospital.

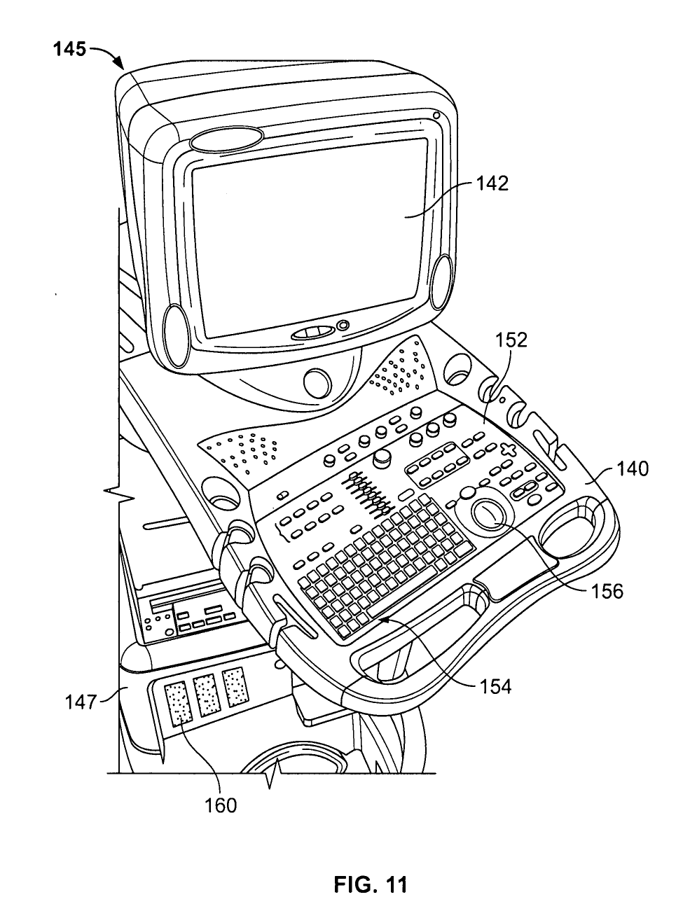

FIG. 11 illustrates a console ultrasound imaging system 145 provided on a movable base 147. The console ultrasound imaging system 145 may also be referred to as a cart-based system. A display 142 and user interface 140 are provided and it should be understood that the display 142 may be separate or separable from the user interface 140. The user interface 140 may optionally be a touchscreen, allowing the operator to select options by touching displayed graphics and icons, as well as drawing the contour and/or selecting points within the image that are used to detect patient anatomy and/or fetal head position. The system 145 has at least one probe port 160 for accepting probes.

The user interface 140 also includes control buttons 152 that may be used to control the portable ultrasound imaging system 145 as desired or needed, and/or as typically provided. The user interface 140 provides multiple interface options that the user may physically manipulate to interact with ultrasound data and other data that may be displayed, as well as to input information and set and change scanning parameters. The interface options may be used for specific inputs, programmable inputs, contextual inputs, and the like. For example, a keyboard 154 and track ball 156 may be provided.

A technical effect of at least one embodiment is the ability to monitor the progress of labor using ultrasound. Another embodiment is to monitor the movement of an object within an ultrasound volume over time. In labor, the position of the fetal head may be monitored with respect to a patient's anatomy, such as with respect to the pubis. Over time, different volumes of ultrasound data are acquired and adjusted to have the same orientation, and thus movement of the fetal head, or lack thereof, may be determined, allowing a determination to be made as to whether additional measures may be desired to assist the labor. The position of the fetal head may be indicated with a contour, an angular relationship between the fetal head and the anatomy, and/or a rotation of a midline defined based on the fetal head, for example.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method for tracking movement of an object within an ultrasound volume, comprising:
acquiring at least first and second volumes of ultrasound data comprising imaging data representing an anatomical structure within a patient and at least a portion of an object, the second volume being acquired at a time subsequent to the first volume;
identifying a first relationship between the object and the anatomical structure on a first image that is based on the first volume;
identifying a second relationship between the object and the anatomical structure on a second image that is based on the second volume; and
determining movement of the object based on at least one of the first and second relationships.

2. The method of claim 1, further comprising:
displaying a landmark on the first image based on the first volume; and
adjusting the first volume based on a relation between the anatomical structure and the landmark.

3. The method of claim 1, further comprising:
displaying a third image that is orthogonal to the first image, the third image being based on the first volume;
displaying a first landmark on the first image and a second landmark on the third image; and
adjusting the first volume based on a first relation between the anatomical structure and the first landmark and a second relation between the anatomical structure and the second landmark.

4. The method of claim 1, further comprising:
wherein the first relationship further comprises identifying a first contour of the object on the first image;
wherein the second relationship further comprises identifying a second contour of the object on the second image; and
the determining further comprising comparing positions of the first and second contours with respect to at least one of each other and the anatomical structure.

5. The method of claim 1, further comprising:
wherein the first relationship further comprises identifying a first angular relationship between the object and the anatomical structure on the first image;
wherein the second relationship further comprises identifying a second angular relationship between the object and the anatomical structure on the second image; and
the determining further comprising comparing the first and second angular relationships with respect to one of each other and the anatomical structure.

6. The method of claim 1, wherein the anatomical structure comprises a pubis and the object comprises a fetal head, wherein the first relationship further comprises:
identifying a first marker parallel to the pubis on the first image;
identifying a second marker on the first image based on a start of a parietal bone of the fetal head, the second marker being further based on a tangent based on the start of the parietal bone; and
determining an angular relationship between the first and second markers.

7. The method of claim 1, wherein the anatomical structure comprises a pubis and the object comprises a fetal head, wherein the first relationship further comprises:
identifying a midline associated with the fetal head on the first image;
identifying a first marker parallel to the pubis on the first image; and
determining an angular relationship between the midline and the first marker.

8. An ultrasound system, comprising:
a transducer acquiring volumes of ultrasound data, the volumes each comprising imaging data representing a symphsis pubis (pubis) within a patient and at least a portion of a fetal head, the volumes being acquired with an elapse in time there-between;
a display for displaying at least one image based on the volumes; and
a user interface for accepting input from an operator, the input being based on at least one of the pubis and the fetal head within the at least one image, the display indicating a relationship of the fetal head to the patient based on the input.

9. The system of claim 8, wherein the display further displaying first and second orthogonal images from a first volume, the system further comprising a processor module configured to display first and, second landmarks on the first and second orthogonal images, respectively, the first volume being adjusted based on anatomy within the patient and the first and second landmarks.

10. The system of claim 8, further comprising a processor module configured to determine an angular relationship between the pubis and the fetal head.

11. The system of claim 8, wherein the display further displaying a first image from the volume, wherein the user interface being further configured to accept at least one point associated with the first image, the system further comprising a processor module configured to determine a contour of at least a portion of the fetal head based on the at least one point.

12. The system of claim 8, wherein the display being configured to display first and second images based on first and second volumes, respectively, the user interface being further configured to accept at least first and second points associated with the fetal head within the first and second images, respectively, the system further comprising a processor module configured to determine first and second contours of at least a portion of the fetal head on the first and second images, respectively, the first and second contours being based on the at least first and second points, the display further displaying the first and second contours simultaneously on at least one of the first and second images.

13. The system of claim 8, wherein the system is one of a handheld system, a portable system, a miniaturized system, and a console-based system.

14. A method for determining progress of a fetus during labor, comprising:
accessing a first volume of ultrasound data comprising imaging data representing a pubis within a patient and at least a portion of a fetal head;
identifying a first relationship between the fetal head and the pubis on a first image that is based on the first volume;
accessing a second volume of ultrasound data comprising imaging data representing the pubis and at least a portion of the fetal head, the second volume being acquired at a time subsequent to the first volume, the first and second volumes being aligned with respect to the pubis;
identifying a second relationship between the fetal head and the pubis on a second image that is based on the second volume; and
determining progress of the fetal head based on at least one of the first and second relationships.

15. The method of claim 14, further comprising:
identifying a first contour of the fetal head on the first image;
identifying a second contour of the fetal head on the second image; and
determining progress of the fetal head based on at least one of the first and second contours.

16. The method of claim 15, further comprising overlaying the first and second contours simultaneously on at least one of the first and second images.

17. The method of claim 15, further comprising displaying the first contour with a first representation and displaying the second contour with a second representation that is different than the first representation.

18. The method of claim 14, further comprising:
acquiring three-dimensional (3D) ultrasound data comprising the pubis and at least a portion of the fetal head;
displaying first and second landmarks on first and second orthogonal images based on the 3D ultrasound data; and
adjusting the 3D ultrasound data based on the first and second landmarks.

19. The method of claim 14, wherein the first and second relationships further comprise first and second angular relationships, respectively, between the fetal head and the pubis on the first and second images, respectively.

20. The method of claim 14, wherein the first and second relationships further comprise first and second midline measurements, respectively, of the fetal head based on the first and second images, respectively.
